(19) United States
(12) Patent Application Publication
(10) Pub. No.: US 2005/0085791 A1
(43) Pub. Date: Apr. 21, 2005

(54) Disinfectant, antibiotic and anesthetic containing device for injections and incisions

(76) Inventors: Sharon M. Shaw, New York, NY (US); Pierre R. Alfred, Amherst, MA (US)

Correspondence Address: PATREA L. PABST;PABST PATENT GROUP LLP, 400 COLONY SQUARE, SUITE 1200, ATLANTA, GA, 30361, US

(21) Appl. No.: 10/943,724

(22) Filed: Sep. 17, 2004

Related U.S. Application Data
(60) Provisional application No. 60/503,597, filed on Sep. 17, 2003.

(51) Int. Cl. A61M 31/00

(52) U.S. Cl. 604/506; 424/426

(57) ABSTRACT

Devices, kits and methods for reducing the risk of infection at an injection or incision site are described herein. The device contains a bioadhesive, biocompatible and bioerodable material and one or more disinfectant agents. In the preferred embodiment, the material is formed of one ore more hydrogels Optionally, the device also contains one or more anesthetics to decrease discomfort. The device may be marked or calibrated to facilitate localized injection or incision at a pre-specified site on the skin or mucus membrane. After identifying or selecting the injection or incision site, the device is placed on the site for a time sufficient to achieve localized disinfection, and optionally localized anesthesia. Then the needle or surgical instrument is inserted through the composition into the site. Thereafter, the drug is administered, fluid is removed, in the case of an injection, or the surgical instrument is placed at the site, in the case of an incision. Then the needle or surgical instrument is removed from the site, and the device forms a continuous seal over the site. The disinfectant and/or anesthetic is delivered before, during, and/or after the treatment. Optionally, the disinfectant and/or anesthetic is released in a controlled-release manner. Optionally, the disinfectant and/or anesthetic may de delivered following a time-delay. In a second embodiment, the device may be placed at a site following the injection or incision at the site to reduce the risk of infection, provide anesthesia, and/or prevent reflux of blood or fluid following the injection or incision.

### CROSS-REFERENCE TO RELATED APPLICATIONS

This application claims priority to U.S. provisional application 60/503,597, filed Sep. 17, 2003.

The present invention is directed at devices for disinfecting injection and incision sites.

### BACKGROUND OF THE INVENTION

Injections and incisions are some of the one of the most common means of both sampling internal bodily fluids/tissues and delivering therapeutic agents. Current clinical standards involve the use of topical liquid disinfectants, such as alcohol, povidone iodine and/or antibiotics, prior to injection so as to minimize the risk of infection. Local anesthetics are not typically used, except in particularly sensitive areas, such as the eye, or on buccal or gastrointestinal mucosa.

Application of topical liquid disinfectants is both imprecise and inconvenient. The duration of action of liquid disinfectant agents is limited in most instances to the time the agent is actually in the liquid phase, which, in turn, is limited by drying time. This method of application does not allow for ongoing disinfection of the site following the injection or incision, unless liquid disinfectant is reapplied. Wide-spread application of liquid disinfectants in this fashion also limits the injector's ability to place the injection in a pre-defined manner without the use of either an indelible marking instrument applied prior to disinfection or a sterilized marking instrument following disinfection. Finally, liquid disinfectant agents do not generally limit efflux of injection material(s) or localized bleeding at the site of injection or incision.

According to statistics from the Center for Disease Control, there are currently 17 million diabetics in the United States. The direct and indirect costs of diabetes have been estimated by the American Diabetes Association to be over 220 billion dollars each year. A large number of diabetics are insulin dependent, and require regular daily transdermal injections of insulin. As diabetics are at increased risk for infection, it is essential that the site of all insulin injections are disinfected prior to injection. Currently, skin disinfection is achieved with liquid disinfectant agents, most notably alcohol swabs. This approach provides limited, poorly localized, and short-acting disinfection. It also provides no means to prevent localized bleeding or bacterial contamination at the injection site upon withdrawal of the needle.

Intraocular injection for vitreous biopsy and/or intravitreous injection of a therapeutic agent such as a drug or gas is a common, but somewhat risky, procedure. First, intraocular infection, or endophthalmitis, can result if the injection site is not adequately prepared and disinfected. Second, injections must be made in an anatomically precise location, measured as 3.5 to 4.0 mm posterior to the surgical limbus (the junction of the cornea and the sclera). The current standard procedure involves applying topical antibiotics and 5% to 10% povidone iodine to the eyelid margin, the eye lashes and the conjunctival surface, measuring with a sterile caliper 3.5 to 4.0 mm posterior to the surgical limbus, applying a sterile cotton-tip swab at the site to minimize the risk of efflux of drug and/or vitreous following the injection, and administering topical antibiotic drops for up to three days following the procedure to minimize the possibility of post-injection infection. The use of so many different devices increases the risk of both misplacing the site of the injection and of developing infection following injection.

25-guage vitrectomy instruments are available and permit direct insertion of the instrument through the conjunctiva and self-sealing sclerostomy incision sites. As with intravitreous injection, the current standard of care for disinfecting the intended incision sites involves applying topical antibiotics and 5% to 10% povidone iodine to the eyelid margin, the eye lashes and the conjunctival surface, measuring with a sterile caliper 3.5 to 4.0 mm posterior to the surgical limbus, applying a sterile cotton-tip swab at the site to minimize the risk of efflux of drug and/or vitreous following removal of the instruments from the sclerostomies, and administering topical antibiotic drops for up to three days following the procedure to minimize the possibility of post-injection infection. In addition, manipulation of vitrectomy instruments across the conjunctival and scleral can produce traumatic tears in overlying conjunctiva, further increasing the risk wound contamination following the procedure.

Modern cataract surgery involves placement of various instruments into the anterior chamber of the eye through small incisions at the surgical limbus. Small numbers of bacteria may be introduced through these incisions resulting in endophthalmitis, both during and for a short time following the surgical procedure until healing of the incision site provides enough structural integrity to prevent bacteria from entering the wound.

Therefore, it is an object of the invention to provide methods for providing anesthesia, reducing the risk of infection, and/or minimizing the movement of fluids at the site of an injection or incision.

It is a further object of the invention to provide devices for providing anesthesia, reducing the risk of infection and/or minimizing the movement of fluids at the site of an injection or incision.

### BRIEF SUMMARY OF THE INVENTION

Devices, kits and methods for reducing the risk of infection at an injection or incision site are described herein. The device contains a bioadhesive, biocompatible and bioerodable material and one or more disinfectant agents. In the preferred embodiment, the material is formed of one ore more hydrogels Optionally, the device also contains one or more anesthetics to decrease discomfort. The device may be marked or calibrated to facilitate localized injection or incision at a pre-specified site on the skin or mucus membrane. After identifying or selecting the injection or incision site, the device is placed on the site for a time sufficient to achieve localized disinfection, and optionally localized anesthesia. Then the needle or surgical instrument is inserted through the composition into the site. Thereafter, the drug is administered, fluid is removed, in the case of an injection, or the surgical instrument is placed at the site, in the case of an incision. Then the needle or surgical instrument is removed from the site, and the device forms a continuous seal over the site. The disinfectant and/or anesthetic is delivered before, during, and/or after the treatment. Optionally, the disinfectant and/or anesthetic is released in a controlled-release manner. Optionally, the disinfectant and/or anesthetic may de delivered following a time-delay. In a second embodiment, the device may be placed at a site following the injection or incision at the site to reduce the risk of infection, provide anesthesia, and/or prevent reflux of blood or fluid following the injection or incision.

### DETAILED DESCRIPTION OF THE INVENTION

I. Device

The device is formed of a polymeric material and a disinfectant or other active ingredient such as an anesthetic. The device is biocompatible, bioadhesive and bioerodible. In the preferred embodiment, the time required for the device to completely erode varies from minutes to days following injection or incision. In the preferred embodiment the erosion time is between approximately one and two days, The device is generally small, less than 10 mm in diameter, but may be larger to suite an intended and specialized use, such as incisions that are greater than 10 mm. The device may be any shape (e.g. square, rectangle, circle, or oval). In one embodiment, the device is a circular or oval disc. The device may be transparent, translucent, or of variable opaqueness. Optionally, the device contains one or more markings to identify the intended site of injection or incision or to assist in measuring the distance from recognized anatomical landmarks, such as the surgical limbus, to the intended site of injection or incision. Optionally, the device is fabricated with two linear or curvilinear depressions on the non-bioadhesive surface to facilitate manipulation and placement using a sterile instrument, such as forceps.

a. Materials

The device is formed from a bioadhesive, biocompatible and bioerodable material. In the preferred embodiment, the device contains one or more hydrogel polymers. These polymers allow for storage and time-dependent release of the disinfectant and/or anesthetic agent(s), are bioadhesive, and bioerode. Suitable hydrogels include film-forming hydrogels and bioadhesive polymers.

Bioadhesive Polymers

Hydrophilic polymers and hydrogels tend to have bioadhesive properties. Hydrophilic polymers that contain carboxylic groups (e.g., poly [acrylic acid]) tend to exhibit the best bioadhesive properties. Polymers with the highest concentrations of carboxylic groups are preferred when bioadhesiveness on soft tissues is desired. Various cellulose derivatives, such as sodium alginate, carboxymethylcellulose, hydroxymethylcellulose and methylcellulose also have bioadhesive properties. Some of these bioadhesive materials are water-soluble, while others are hydrogels. Rapidly bioerodible polymers such as poly (lactide-co-glycolide), polyanhydrides, and polyorthoesters, whose carboxylic groups are exposed on the external surface as their smooth surface erodes, can also be used to form bioadhesive materials. In addition, polymers containing labile bonds, such as polyanhydrides and polyesters, are well known for their hydrolytic reactivity. Their hydrolytic degradation rates can generally be altered by simple changes in the polymer backbone. Upon degradation, these materials also expose carboxylic groups on their external surface, and accordingly, these can also be used for bioadhesive drug delivery systems.

Hydrogels

Suitable hydrogels can be formed from synthetic polymers such as polyethylene glycol, polyethylene oxide, polyvinyl alcohol, polyvinyl pyrrolidone, polyacrylates, poly (ethylene terephthalate), poly (vinyl acetate), and copolymers and blends thereof, as well as natural polymers such as cellulose and alginate, as described above. Exemplary materials include SEPRAFILM® (modified sodium hyaluronate/carboxymethylcellulose, Genzyme Pharmaceuticals) and INTERCEED® (oxidized regenerated cellulose, Johnson & Johnson Medical, Inc.), BEMA® (Bioerodible Muco-Adhesive Disc, Atrix Laboratories, Inc.), and Bioadhesive Ophthalmic Drug Insert (BODI) (described in Gurtler F, et al., J. Controlled Release 1995; 33:231-236). The use of hydrogels to provide local delivery of drugs is described, for example, in U.S. Pat. No. 5,410,016 to Hubbell et al.

b. Disinfectant

The disinfectant may be an alcohol (such as ethyl alcohol, methyl alcohol, isopropyl alcohol, butyl alcohol, or propyl alcohol), iodine or povidone iodine; an alkali disinfectant (such as sodium hydroxide or calcium oxide), a biguanide disinfectant such as chlorhexidene, Virosan (a bovine antiseptic and bactericidal udder creme), or Nolvasan® (American Home Products Corp.), a cationic surfactant (such as Parvosol® (Chlorhexidine, Hess & Clark, Inc.), Roccal-D® Plus, A33® (Quaternary Ammonium Disinfectant,), Brulin Maxima 128 (quaternary germicidal detergent), Bramton Ken Care Disinfectant, Unicide 256 Germicidal Detergent (quaternary ammonium compound), benzalkonium chloride, bensathonium chloride, or cetylpyridinum chloride), a halogen containing compound (such as sodium hypochlorite, alcide, sodium dichloroisocyanurate, calcium hypochlorite, or organic chloride), an oxidizing peroxide (such as hydrogen peroxide, sodium perborate, benzolyl peroxide, or potassium permanganate), a phenol or related compound (such as carbolic acid or cresylic acid), a synthetic phenol (such as chlorosyenols, hexachlorophene, sporicidin, parachlorometaxylenol, or dichlorometaxylenol), a reducing agent or aldehyde (such as glutaraldehyde, formalin, Cidex® (Johnson & Johnson Corp.), or Wavicide®-01 (Wave Energy Systems Inc.)), thimerosal, an antimicrobial agent (such as erythromycin, tetracycline) or combinations thereof. Disinfectants are usually toxic at high concentrations. Useful concentrations are known to those skilled in the art, or readily discernible using standard assay techniques.

c. Anesthetic

An anesthetic may be included in the device to reduce discomfort associated with the injection or incision. Suitable anesthetics include, but are not limited to, local anesthetics, such as lidocaine, tetracaine (amethocaine), prilocaine, benzocaine, bupivacaine, cocaine, etidocaine, mepivacaine, pramoxine, prilocaine, procaine, proparacaine, ropivacaine, and mixtures thereof. Typical dosages include 2% to 10% (wt/wt) lidocaine or 2% (wt/wt) tetracaine, although considerably less anesthetic may be used. Two or more anesthetics may be combined to allow for optimal pharmacokinetic release and/or to enhance penetration across the skin or mucus membrane.

In one embodiment, the device contains a combination of a disinfectant and an anesthetic, for example: (a) benzalkonium chloride or thimerosal, with or without (b) povidone iodine, and (c) lidocaine or tetracaine. The amounts of each disinfectant or anesthetic agent in the device may vary with each application, and can be determined empirically. For example, the combination could contain 70% (wt/wt) ethyl alcohol or 5-10% (wt/wt) povidone iodine and 2-4% (wt/wt) lidocaine or 2% (wt/wt) tetracaine. Another suitable combination contains 70% ethyl alcohol or 5-10% (wt/wt) povidone iodine, 2-4% (wt/wt) lidocaine, and 2-4% prilocaine.

d. Other Biologically Active Agents

Optionally, the device contains additional biologically active agents, such as antimicrobial fungicides or virocides. Optionally, the device also contains an antibiotic, such as, but not limited to, amoxicillin, ampicillin, cefaclor, clarithromycin, ceftriaxone, cefprozil, gentamicin sulfate, and vancomycin.

e. Excipients and Additives

The device may include a pharmaceutically acceptable diluent or carrier and other excipients. The device may include agents that reduce irritation, such as glycerin, petrolatum jelly, petrolatum, mineral oil, ethylene glycol, and glycerol, and combinations thereof. Optionally the device contains pH stabilizers or buffers, which optimize and stabilize the pH of the skin or mucus membrane. Optionally the device contains one or more lectins to enhance bioadhesion.

II. Methods of Using Disinfectant Device

The disinfectant device can be used in many different applications to reduce the risk of infection and to prevent bleeding or loss of fluids following injection or incision. Suitable applications include drug injections, fluid removal, and eye surgery, such as a vitrectomy and cataract removal. The device is placed on the skin or a mucus membrane. In the preferred embodiment, the device is applied to the skin, such as before a patient is injected with insulin. In another embodiment, the device is applied to a site prior to an injection in the eye or across another mucus membrane. In still another embodiment, the device is placed at the across the incision at corneal limbus before, during, or after vitreous or cataract surgery. The device is left in place for a time period sufficient to disinfect, and optionally to anesthetize, the site. In one embodiment, the device is left in place for a time period ranging from five to fifteen minutes, during which it completely erodes. In another embodiment, the device is left in place for one to three days. In each case, the device remains in place until it has completely eroded.

After placing the disinfectant device on the predetermined site, a needle, cannula, or other instrument, such as a surgical instrument, is inserted into and through the device and into the skin or mucus membrane. In one embodiment, the instrument is an ocular surgery instrument used in cataract surgery, glaucoma surgery or vitreoretinal surgery. Optionally, the device self-seals upon removal of the needle or surgical instrument. The device is not removed from the site, rather it adheres to the site and degrades over a period of time ranging from minutes to days.

While the disc remains on the site, the disinfectant and/or antimicrobial agent is released. Disinfectant and/or antimicrobial agents can be delivered for a time period ranging from minutes to days. In one embodiment, the disinfectant, anesthetic and/or antimicrobial agent is delivered for less than 24 hours, or for the length of time required for the device to degrade.

The devices may contain anesthetic, which is delivered to the patient for a time period ranging from 5 to 60 minutes. In one embodiment, the anesthetic is delivered before the injection. Depending on the application, the disinfectant and/or anesthetic may be delivered before, during, and/or after the injection.

Transdermal Injection of Insulin

Prior to the injection of a drug, such as insulin, the disinfecting device is placed at the intended injection site. After a period of time effective to disinfect and anesthetize the injection site, which generally ranges from 5 to 60 minutes, an insulin injection is given through the device, preferably through the center which is marked to facilitate placement. The disinfecting device may be in the shape of a circular disc, optionally with a clearly marked center to allow for pre-determined identification of the intended injection site. The device may be self-sealing, thereby preventing bleeding upon withdrawal of the needle. The precise combination of disinfectants, anesthetic agent, and/or antimicrobial agent can be determined empirically; similarly, the time-release and bioerosion characteristics of the device can be determined empirically using routine techniques.

Transconjunctival Injection and/or Intravitreous Injection of Drug

Prior to a transconjunctival injection for a vitreous biopsy and/or intravitreous injection of drug, a disinfecting device is placed at the intended injection site. Optionally, the device is a bioadhesive circular disc, with a clearly marked center for injection 3.5 to 4.0 mm from the disc edge, thereby eliminating the need for a sterile measuring device, such as a caliper. Placement of the edge of the disc at the surgical limbus will precisely identify the intended injection site, thereby obviating the need for sterile calipers. The disc may also be self-sealing, thereby obviating the need for a sterile cotton-tip swab to prevent reflux of drug and/or vitreous, and for post-injection antibiotic drops. The precise combination of disinfectants, anesthetic agent, and/or antimicrobial agent can be determined empirically; similarly, the time-release and bioerosion characteristics of the device can be determined empirically, using routine techniques.

Vitrectomy

To assist in a vitrectomy, the disinfecting device is placed at the intended site of insertion of a 25-guage vitrectomy instrument. The 25-guage vitrectomy instrument is directly inserted through the conjunctiva to remove the vitreous. The disinfecting device may be in the shape of a circular disc, optionally with a clearly marked center to allow for pre-determined identification of the intended insertion site. Placement of the edge of the disc at the surgical limbus could be used to precisely identify the intended injection site, thereby obviating the need for a sterile caliper. Optionally, the device contains a local anesthetic agent to provide anesthesia at the site of the insertion of the 25-guage vitrectomy instrument. The disc may also be self-sealing, thereby obviating the need for a sterile cotton-tip swab to prevent reflux of drug and/or vitreous.

Optionally, the device contains a sustained released disinfectant or antimicrobial agent, thereby obviating the need for post-injection antibiotic drops. The precise combination of disinfectants, anesthetic agent, and/or antimicrobial agent can be determined empirically; similarly the time-release and bioerosion characteristics of the device can be determine empirically using routine techniques.

Anterior Segment Surgery

The device may be used on the eye before, during or after anterior segment surgery, such as cataract surgery, intraocular lens placement or exchange, or glaucoma surgery. In one such application, the device is applied prior to the surgery, before the needle or other instrument is inserted in the anterior segment of the eye. In another application, the device is applied to injection or incision site immediately following the surgical procedure. The precise combination of disinfectants, anesthetic agent, and/or antimicrobial agent can be determined empirically; similarly the time-release and bioerosion characteristics of the device can be determined empirically using routine techniques.

While the specific combination of alcohol, iodine, povidone iodine, antibiotics, and/or other disinfecting agents are indication-specific and determined empirically, as are the time-release and bioerosion characteristics of the device, the general properties of the disinfecting device are the same as described above for intravitreal injection or insertion of vitrectomy instruments. In the preferred embodiment, the disinfectant is a combination of: (a) 5-10% (wt/wt) povidone iodine, (b) 2-4% (wt/wt) lidocaine or 2% (wt/wt) tetracaine, and (c) an antibiotic. The ethyl alcohol releases immediately upon application, over a time period of five to fifteen minutes. The antibiotic releases as the device erodes, over a period of time ranging from one to three days.

Those skilled in the art will recognize, or be able to ascertain using no more than routine experimentation, many equivalents to the specific embodiments of the invention described herein. Such equivalents are intended to be encompassed by the following claims.

III. Kits

Kits for use during surgery or injections include the bioadhesive, bioerodible device and a surgical or an injection instrument. Suitable instruments include ocular surgery instruments for cataract surgery, glaucoma surgery or vitreoretinal surgery, needles, and cannulas.

1. A device comprising a bioadhesive, biocompatible and bioerodable material and one or more disinfectants, antibiotics, anesthetics, or combinations thereof.

2. The device of claim 1, comprising a disinfectant selected from the group consisting of alcohols, iodine or povidone iodine; alkali disinfectants, biguanide disinfectants, cationic surfactants, halogen containing compounds, oxidizing peroxides, phenols, reducing agents, aldehydes, thimerosal, antimicrobial agents and combinations thereof.

3. The device of claim 1, wherein the bioadhesive, biocompatible and bioerodable material is a hydrogel.

4. The device of claim 1, further comprising one or more pH stabilizers or buffers.

5. The device of claim 1, further comprising an additive selected from the group consisting of glycerin, petrolatum jelly, petrolatum, mineral oil, ethylene glycol, and glycerol, and combinations thereof.

6. The device of claim 1, comprising an anesthetic selected from the group consisting of lidocaine, tetracaine, prilocaine, benzocaine, bupivacaine, cocaine, etidocaine, mepivacaine, pramoxine, prilocaine, procaine, proparacaine, ropivacaine, and mixtures thereof.

7. The device of claim 1 comprising an antibiotic.

8. A method for administering a disinfectant, antibiotic, anesthetic or combination thereof to an injection or incision site comprising applying a device to the injection site, wherein the device comprises a bioadhesive, biocompatible and bioerodable material, and a compound selected from the group consisting of one or more disinfectants, one or more antimicrobial agents, and one or more local anesthetics, and combinations thereof; and
inserting an injection or surgical instrument into the device.

9. The method of claim 8, wherein the device further comprises a marking identifying the location for the injection or incision site.

10. The method of claim 9, wherein the injection or incision instrument is inserted in the marking.

11. The method of claim 8, wherein the disinfectant is selected from the group consisting of alcohols, iodine or povidone iodine; alkali disinfectants, biguanide disinfectants, cationic surfactants, halogen containing compounds, oxidizing peroxides, phenols, reducing agents, aldehydes, thimerosal, antimicrobial agents and combinations thereof.

12. The method of claim 8, wherein the device self-seals upon removal of the injection instrument.

13. The method of claim 8, wherein the disinfectant is delivered to the injection site over a period of time ranging from one to three days.

14. The method of claim 8, wherein the disinfectant is delivered to the injection or incision site after the injection.

15. The method of claim 8, wherein the injection site is located on the skin or a mucus membrane.

16. The method of claim 15, wherein the injection or incision site is located on a mucus membrane in the eye.

17. A kit for administering a disinfectant, antibiotic, anesthetic, or combination thereof to an injection or incision site comprising a device, wherein the device comprises a bioadhesive, biocompatible and bioerodable material, one or more disinfectants, one or more antimicrobial agents, and one or more local anesthetics; an injection or surgical instrument.

18. The kit of claim 17, wherein the instrument is selected from the group consisting of ocular surgery instruments for cataract surgery, glaucoma surgery or vitreoretinal surgery, needles, and cannulas.
